(19) United States
(12) Patent Application Publication
(10) Pub. No.: US 2002/0122800 A1
(43) Pub. Date: Sep. 5, 2002

(54) Treatment of inflammatory oral diseases with a combination of inhibitors of TNF-alpha and immunosuppressive agents

(76) Inventor: Stephen T. Sonis, Wayland, MA (US)

Correspondence Address: Paul T. Clark, Clark & Elbing LLP, 101 Federal Street, Boston, MA, 02110, US

(21) Appl. No.: 10/005,642

(22) Filed: Dec. 5, 2001

Related U.S. Application Data
(60) Provisional application No. 60/251,736, filed on Dec. 5, 2000.

(51) Int. Cl. A61K 39/395; A61K 31/56

(52) U.S. Cl. 424/145.1; 514/171

(57) ABSTRACT

The invention provides methods for treating inflammatory oral diseases, involving administration of combinations of inhibitors of TNF-α and immunosuppressive agents.

### BACKGROUND

Inflammatory oral diseases are the most common mouth diseases in North America. Included in these are recurrent aphthous stomatitis (RAS) and oral lichen planus (OLP) mucosa. RAS affects up to 66% of adults, with an overall prevalence of 20%. One to 2% of adults reportedly suffer from OLP.

The clinical presentations of RAS and OLP are quite different. RAS is characterized by one or more painful ulcers of the movable oral mucosa that typically last 7 to 10 days. The patient then undergoes a period during which he/she is symptom free. The frequency of recurrence is variable, but usually ranges from days to months. RAS occurs in three forms: minor RAS, major RAS, and herpetiform ulcers. Over half of patients with RAS develop the minor type. Twenty-two percent are affected by major lesions.

Patients with minor RAS typically develop lesions every few months. Patients may experience a prodromal stage during which they note localized burning or itching of the mucosa a day or two before ulceration occurs. The ulcers that develop in minor RAS are usually oval, with a diameter of a centimeter or less. Despite their relatively small size, the lesions are extremely uncomfortable and are especially bothersome if they occur in an area of the mouth that is subject to functional trauma. The center of the ulcer is yellowish-gray in color and is surrounded by an erythematous ring. Healing usually occurs in 7 to 10 days without scarring.

Oral lichen planus typically has a later age of onset than RAS. Unlike, RAS in which patients have recurrent ulcerations between which there are periods of no lesions, OLP tends to be more chronic, although exacerbations and remissions may occur. OLP may have one of three presentations. The most common form involves areas of linear, papillated, hyperkeratosis that forms the typical reticulated pattern (Wickam's striae) that are associated with OLP. While these lesions may create a feeling of mucosal roughness for the patient, they are generally not painful. In contrast, erosive lichen planus, the next most common variation results in erosion of the mucosal surface such that there is detachment of the epithelium. Striated lesions are often contiguous. The erosive nature of these lesions is painful and sore. The most dramatic form of OLP is the blistering or bullous form in which patients develop vesiculobullous lesions comparable to those seen in pemphigoid.

Despite their disparate clinical course and appearance, RAS and OLP share some similarities in their etiology. Both conditions appear to be of the result of immunopathogenic processes. For example, patients with RAS may have increased levels of peripheral blood CD8+ and reduced CD4+ lymphocytes. This reversal in CD4 and CD8 ratios is most dramatic among patients with severe RAS. Additionally, the ulcerative phase of RAS is associated with the appearance in the mucosa of cytotoxic lymphocytes. Finally, increased T cell receptor-γδ+ is seen in patients with active RAS compared to patients with inactive RAS or in patients who do not have the condition.

There is significant clinical and experimental evidence to support an immunologic pathogenesis for OLP. Histologically, OLP is characterized by a cellular infiltrate that is rich in T-cells, macrophages and mast cells. These cells secrete proinflammatory cytokines that modify the course of the disease. It is believed that OLP is a T-cell mediated process.

In addition, it appears that the pro-inflammatory cytokine, tumor necrosis factor-alpha (TNF-α), plays a role in the pathogenesis of both conditions. RAS has been treated with thalidomide, a TNF antagonist.

Both RAS and OLP have been treated with systemic and topical steroid therapy. Topical steroids for RAS and OLP have included fluocinonide (Lidex), Clobetasol proprionate (Temovate), Halbetasol proprionate (ultravate), Triamcinaolone acetonide (Kenalog), and Dexamethasone (Decadron). These agents have been used as ointments, gels, or rinses, and result in a reduction of symptoms because of their anti-inflammatory effect. There is no evidence to suggest that they ameliorate the severity of RAS ulceration or prevent its formation. It is questionable as to whether the duration of ulceration is affected. Topical steroids have provided the basis for the management of erosive lichen planus. More aggressive steroid or immunomodulatory therapy has been prescribed for bullous lesions. In general, topical steroids may control symptoms and reduce the severity of lesions.

The only approved medication for the topical treatment of RAS is amlexanox, a leukotriene inhibitor.

No combination products exist to treat either RAS or OLP.

### SUMMARY OF THE INVENTION

I have discovered that combination therapy can be an effective treatment for RAS and OLP. The treatment involves administering to the patient two therapeutic agents: the first is an immunosuppressive agent, and the second is a compound that exerts activity against TNF, either by blocking or antagonising the TNF receptor, or, more preferably, interferring with the production of TNF, e.g., by down-regulating transcription of the TNF gene. Both agents can be administered either topically or systemically; most preferably, both are administered topically to the lesion itself, in a carrier such as a rinse or gel. Preferred immunosuppressive agents are topical steroids or known immunosuppressive agents such as cyclosporin, FK 506, Decadron, and triamcimalone acetonide. Anti-TNF agents include thalidomide and Pentoxifylline (PTX): 3,7-Dihydro-3,7-dimethl-1-(5-oxohexyl)-1H-purine-2,6-dione, which is an analog of methylxanthine theobromine. PTX, which was initially developed as an agent for the treatment of peripheral vascular disease, exerts cellular effects on platelets, endothelial cells, neutrophils, and macrophages; PTX also is known to have activity against TNF and interlukin-1. It appears to act by down-regulating transcription of the TNF gene.

The components of the combination therapy of the invention are used in amounts in ranges for which these agents are used to treat other medical conditions in human patients. As discussed above, topical treatment is preferred for local control of disease, so that the risk of unwanted side-effects associated with systemic administration is minimized.

Combination therapies of the invention include the following:

1) A topical ointment or gel consisting of fluocinonide and PTX.

2) A topical rinse consisting of Decadron and PTX

3) A topical rinse consisting of cyclosporin and PTX

4) A topical gel or ointment consisting of triamcinalone acetonide and thalidomide

5) A topical gel or rinse consisting of Decadron and thalidomide.

### CLINICAL EXAMPLE

There follows a description of the successful treatment of a human patient according to the invention.

The patient was a healthy male in his fifties who had longstanding major RAS. The ulcers were located throughout the patient's mouth and oropharynx. Prior to being treated according to the invention, this patient had consulted a number of dental and medical specialists and had had a biopsy performed on a large lesion affecting the tonsillar pillars. At the time of his initial visit with the present inventor, the patient had a number of large ulcerations, the most serious of which was located on the posterier border of the soft palate. The patient, prior to this visit, had been treated with numerous forms of conventional therapy, including systemic steroids and other pharmaceutical agents. All treatements had failed.

The patient was treated according to the invention as follows. The patient was given PTX tablets (one 400 mg tablet per day), combined with a rinse of the topical steroid Decadron. This treatement completely resolved all of the patient's major lesions, after which time the patient was placed on a topical regimen pursuant to which he treats invidividual lesions with the Decadron rinse. The patient reported, approximately six months after initial treatment, that he was completely without mouth ulcers for the first time in eight years.

1. A method of treating a patient suffering from aphthous, said method comprising orally applying to said aphthous a composition consisting essentially of a steroid and an inhibitor of TNF-α.
